(19) United States
(12) Patent Application Publication
(10) Pub. No.: US 2026/0204392 A1
(43) Pub. Date: Jul. 16, 2026

(54) METHOD AND APPARATUS FOR ASSISTING PSYCHOLOGICAL CARE THROUGH MULTIMODAL DATA ANALYSIS BASED ON LARGE MULTIMODAL MODEL

(71) Applicant: KOREA UNIVERSITY RESEARCH AND BUSINESS FOUNDATION, Seoul (KR)

(72) Inventors: Young-gun Ko, Seoul (KR); Jin-young Kim, Seoul (KR); Haewon Min, Seoul (KR); Wonmo Kang, Seoul (KR)

(21) Appl. No.: 19/435,462

(22) Filed: Dec. 29, 2025

(30) Foreign Application Priority Data
Dec. 30, 2024 (KR) 10-2024-0200957

(51) Int. Cl. G16H 20/70; G16H 40/63

(52) U.S. Cl. CPC G16H 20/70; G16H 40/63

(57) ABSTRACT

A psychological care assistant apparatus according to an embodiment may perform: an operation of acquiring basic information of a client; an operation of acquiring biometric information of the client; an operation of generating evaluation information about a psychological state of the client by utilizing an LMM model on the basis of the basic information and biometric information; an operation of providing feedback information to the client or a counselor counseling the client on the basis of the evaluation information; and an operation of acquiring an additional input on the counselor, and generating information on a psychological counseling or treatment plan by inputting a command corresponding to the additional input and the evaluation information into the LMM model.

### CROSS REFERENCE TO RELATED APPLICATION

The present application claims the benefit of Korean Patent No. 10-2024-0200957 filed in the Korean Intellectual Property Office on Dec. 30, 2024, the entire contents of which are incorporated herein by reference.

### TECHNICAL FIELD

The present invention relates to a technique of psychological care, and more particularly, to a technique of accurately evaluating the psychological state of a client and supporting customized counseling and treatment by comprehensively utilizing various kinds of information of the client on the basis of a Large Multimodal Model (LMM) model.

### BACKGROUND OF THE INVENTION

Psychological care is a therapeutic process performed through interactions between a client and a counselor to alleviate psychological difficulties or mental health problems. The object of the psychological care is to alleviate emotional pains, behavioral problems, cognitive distortions, difficulties in human relationships, and the like and improve quality of life of the client.

Conventional psychological care has been accomplished mainly based on self-report psychological tests and interviews with a counselor. In this process, the client provides data in a way of describing his or her psychological state by himself or herself or answering questions, and the counselor analyzes the psychological state and performs counseling on the basis of the data.

Although existing methods of psychological care have advantages of grasping the psychological state in a relatively simple and rapid way, there are some limitations. First, as the self-report psychological test relies on subjective determination of a client, accuracy of the test may be lowered. For example, when the client attempts to distort or conceal his or her state, the diagnosis result may lose reliability.

In addition, the existing methods of psychological care has a tendency of greatly relying on subjective determination of a counselor. This means that consistency of diagnostic results may vary according to the experience and skill level of the counselor. According thereto, quality of psychological care service is inconsistent, and particularly, it is difficult for the client to be provided with appropriate psychological support in a region where the number of counselors is insufficient.

According to the background as described above, improvement in the technique of psychological care is required. Particularly, in addition to the self-report psychological test, it is required to develop a technique of presenting a personalized counseling and treatment plan by evaluating the psychological state of a client from various viewpoints by utilizing more diverse information, and analyzing the psychological state of the client more precisely by comprehensively utilizing verbal and nonverbal data.

### PRIOR ART

### Patent Document

Korean Patent Publication No. 10-2023-0161183

### DISCLOSURE

### Technical Problem

Therefore, the present invention has been made in view of the above problems, and it is an object of the present invention to propose a technique of accurately evaluating a psychological state by comprehensively utilizing various kinds of information of a client, identifying high-risk psychological groups in an early stage, and providing customized psychological care. To this end, it is desired to provide a technique of deriving correlations between verbal and nonverbal data by analyzing basic information and biometric information of the client using a Large Multimodal Model (LMM), and evaluating and predicting a psychological state more precisely.

In addition, the present invention is designed to assist works of a psychotherapist, provide meaningful feedback to a client in real time, and automatically generate an appropriate psychological counseling and treatment plan when needed. It is desired to effectively support promotion of psychological health and early intervention by detecting psychological resistance or problematic behaviors of the client and suggesting a customized intervention plan.

Furthermore, another object of the present invention is to significantly improve the efficiency and accessibility of psychological care service by implementing a highly reliable system that does not rely on subjective determination of a counselor.

Meanwhile, the technical problems of the present invention are not limited to the technical problems mentioned above, and unmentioned other technical problems can be clearly understood by those skilled in the art from the following description.

### Technical Solution

To accomplish the above objects, according to an aspect of the present invention, there is provided a method performed by a psychological care assistant apparatus operated by a processor, the method comprising: an operation of acquiring basic information of a client; an operation of acquiring biometric information of the client; an operation of generating evaluation information about a psychological state of the client by utilizing an LMM model on the basis of the basic information and biometric information; an operation of providing feedback information to the client or a counselor counseling the client on the basis of the evaluation information; and an operation of acquiring an additional input on the counselor, and generating information on a psychological counseling or treatment plan by inputting a command corresponding to the additional input and the evaluation information into the LMM model.

In addition, the basic information may include personal information of the client, self-report psychological test results, and lifelog data.

In addition, the biometric information may include physiological information, behavioral information, vocal information, and verbal information.

In addition, the operation of acquiring biometric information may include an operation of storing a psychological factor of the client, which is determined by classifying a numerical or classification value of physiological information, behavioral information, vocal information, or verbal information corresponding to the biometric signal according to predefined criteria, to be mapped to the biometric information.

In addition, the operation of acquiring biometric information may include an operation of determining inconsistency between at least two pieces of information, among the physiological information, behavioral information, vocal information, and verbal information, corresponding to the biometric signal, determining that the acquired biometric signal is caused by a psychological resistance or false report of the client when the determined inconsistency exceeds a predetermined threshold, and reacquiring the biometric signal of the client.

In addition, the LMM model may generate vector information combining the basic information and the biometric information on the basis of a self-attention mechanism or a cross-attention mechanism, and generates evaluation information about the psychological state of the client on the basis of the combined vector information.

In addition, the operation of generating evaluation information may include: an operation of generating evaluation information about the psychological state of the client on the basis of the self-attention mechanism between text specifying the basic information and text defining the determined psychological factor; and an operation of generating evaluation information about the psychological state of the client on the basis of the cross-attention mechanism between text specifying the basic information and an image or a video specifying the biometric information.

In addition, the feedback information may include temporal tendency in a change of the psychological state of the client and a warning about a psychologically high-risk group.

In addition, the additional input may include information on a current psychological state, details of previous counseling, and a result of observing a specific behavior or response, and the command corresponding to the additional input includes a command text for a specific task that the LMM model should perform on the basis of the additional input.

According to another embodiment, there is provided a psychological care assistant apparatus comprising: a memory including instructions; and a processor that performs a predetermined operation based on the instructions, wherein the operation of the processor includes: an operation of acquiring basic information of a client; an operation of acquiring biometric information of the client; an operation of generating evaluation information about a psychological state of the client by utilizing an LMM model on the basis of the basic information and biometric information; an operation of providing feedback information to the client or a counselor counseling the client on the basis of the evaluation information; and an operation of acquiring an additional input on the counselor, and generating information on a psychological counseling or treatment plan by inputting a command corresponding to the additional input and the evaluation information into the LMM model.

### Advantageous Effects

As the correlation between verbal and nonverbal data is derived by comprehensively analyzing basic information and biometric information of a client, the present invention may enhance efficiency of the psychological health care by grasping psychological problems of the client in an early stage and implementing preventive interventions.

In addition, the present invention reduces the burden of work of psychotherapists, and expands accessibility to psychological care services by automatically generating a counseling and treatment plan customized for a client. Accordingly, as the dependency on the subjective determination of a counselor is lowered, and objective evaluation based on data is allowed, reliability and accuracy of psychological care service can be improved significantly.

Furthermore, the present invention may provide an environment in which a client may receive more continuous and personalized psychological support through automation and advancement in the techniques of psychological care, and practically contribute to promotion of psychological health and improvement of quality of life.

Meanwhile, the effects of the present invention are not limited to those mentioned above, and unmentioned other technical effects will be clearly understood by those skilled in the art from the following descriptions.

### BRIEF DESCRIPTION OF THE DRAWINGS

FIG. 1 is a view showing the configuration of a psychological care assistant apparatus according to an embodiment.

FIG. 2 is a flowchart illustrating the steps of operation performed by a psychological care assistant apparatus according to an embodiment.

FIGS. 3 to 6 are graphs showing values of change measuring the emotional change, facial movement, eye movement, and mouth movement of a client from a specific sentence.

FIG. 7 is an exemplary view showing an operation of classifying the percentile of a value derived from a biometric signal of a client into an upper or lower level according to an embodiment.

FIG. 8 is a view showing the concept of interactions between a psychological care assistant apparatus and a client and a counselor in an initial intake interview situation in the psychological counseling and treatment field according to an embodiment.

FIG. 9 is a view showing the concept of interactions between a psychological care assistant apparatus and a client and a counselor in a situation of assisting a therapeutic expert of individual and group psychological counseling (referred to as a “counselor” in the specification) according to an embodiment.

FIG. 10 is a view showing the concept of interactions between a psychological care assistant apparatus and a client and a counselor in a situation of assisting a therapeutic supervisor of individual and group psychological counseling (referred to as a “counselor” in the specification) according to an embodiment.

FIG. 11 is a view showing the concept of interactions between a psychological care assistant apparatus and a client and a counselor in a situation of assisting a therapeutic expert of individual and group psychological counseling (referred to as a “counselor” in the specification) according to an embodiment.

FIG. 12 is a view showing the concept of interactions between a psychological care assistant apparatus and a client and a counselor in a situation of assisting a health checkup specialist (referred to as a “counselor” in the specification) according to an embodiment.

### DETAILED DESCRIPTION OF THE INVENTION

Details of the objects and technical configurations of the present invention and operational effects according thereto will be more clearly understood by the following detailed description based on the drawings attached in the specification of the present invention. An embodiment according to the present invention will be described in detail with reference to the accompanying drawings.

The embodiments disclosed in this specification should not be construed or used as limiting the scope of the present invention. For those skilled in the art, it is natural that the description including the embodiments of the present specification have various applications. Accordingly, any embodiments described in the detailed description of the present invention are illustrative for better describing of the present invention, and are not intended to limit the scope of the present invention to the embodiments.

The functional blocks shown in the drawings and described below are merely examples of possible implementations. Other functional blocks may be used in other implementations without departing from the spirit and scope of the detailed description. In addition, although one or more functional blocks of the present invention are expressed as separate blocks, one or more of the functional blocks of the present invention may be combinations of various hardware and software configurations that perform the same function.

In addition, the expressions including certain components are expressions of “open type” and only refer to existence of corresponding components, and should not be construed as excluding additional components.

Furthermore, when a certain component is referred to as being “connected” or “coupled” to another component, it may be directly connected or coupled to another component, but it should be understood that other components may exist in between.

Hereinafter, various embodiments of the present invention will be described with reference to the accompanying drawings. However, it should be understood that this is not intended to limit the present invention to specific embodiments, but to include various modifications, equivalents, and/or alternatives of the embodiments of the present invention.

The present invention proposes a psychological care assistant apparatus 100 that implements a technique of accurately evaluating a psychological state and supporting customized counseling and treatment by comprehensively utilizing various information of a client on the basis of a Large Multimodal Model (LMM) model.

Hereinafter, the configuration of the psychological care assistant apparatus 100 of the present invention and the operation of each component will be described.

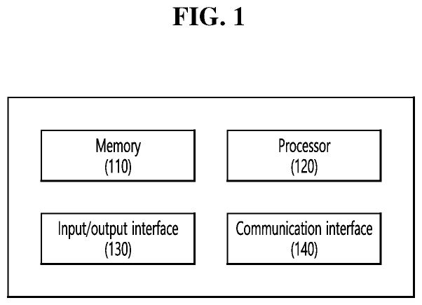

FIG. 1 is a view showing the configuration of a psychological care assistant apparatus 100 according to an embodiment (hereinafter, referred to as an ‘apparatus 100’).

Referring to FIG. 1, the apparatus 100 according to an embodiment may include a memory 110, a processor 120, an input/output interface 130, and a communication interface 140.

The memory 110 may store data acquired from an external device or data generated by itself. The memory 110 may store instructions that may perform the operation of the processor 120. For example, the memory 110 may store the basic information, biometric information, LMM model, and the like of a client that appear in the details of operation described below.

The processor 120 is a computing device that controls the overall operation. The processor 120 may execute instructions stored in the memory 110. The operation of a user terminal 10 and the apparatus 100 according to the embodiment of this document may be understood as an operation performed by the processor 120.

The input/output interface 130 may include a hardware interface or a software interface for inputting or outputting information.

The communication interface 140 allows to transmit and receive information through a communication network. To this end, the communication interface 140 may include a wireless communication module or a wired communication module.

The apparatus 100 may be implemented in various types of apparatuses that can perform an operation through the processor 120 and transmit and receive information through a network. For example, although the apparatus may be implemented as a server, a computer apparatus, a portable communication apparatus, a smart phone, a portable multimedia apparatus, a laptop computer, a tablet PC, or the like, it is not limited to these examples.

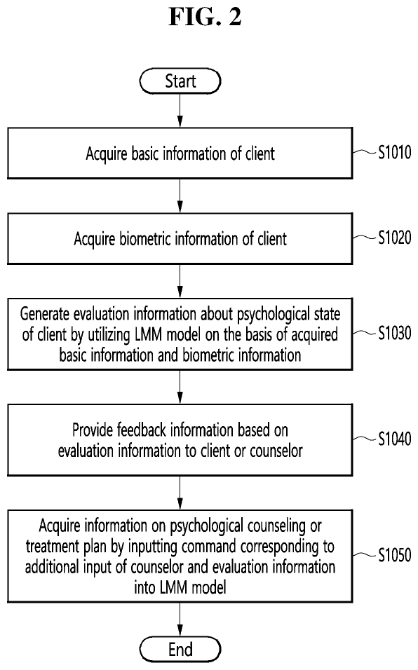

FIG. 2 is a flowchart illustrating the steps of operation performed by the apparatus 100 according to an embodiment. The operation of the apparatus 100 according to the embodiment of FIG. 2 may be understood as an operation performed by the processor 120.

Meanwhile, each step disclosed in FIG. 2 is merely a preferred embodiment in achieving the objects of the present invention, and some steps may be added or deleted as needed, and any one step may be included and performed in another step. The order of each operation disclosed in FIG. 2 is arranged only for convenience of understanding, and this order is not limited to a time-series order, and the order may be changed to operate in a different way according to the choice of the designer.

Referring to FIG. 2, at step S1010, the apparatus 100 may acquire basic information of a client.

The client is an individual participating in psychological care service using the apparatus 100, and means a subject who participates in psychological care through face-to-face or non-face-to-face counseling with a counselor to improve or manage his or her psychological state.

That is, the client himself or herself may see the counselor face-to-face and perform psychological care, and in the case of face-to-face counseling, the counselor may collect information on the client using the apparatus 100, analyze the psychological state of the client, and support counseling on the basis of the psychological state.

In addition, the client may perform non-face-to-face counseling with the counselor, and both the client and counselor may perform psychological care through their own devices (e.g., computers, tablet PCs, smartphones, etc.). In the case of non-face-to-face counseling, the apparatus 100 may operate as a server to support the client and counselor to access non-face-to-face with their own devices, and transmit and receive information with the devices of the client and counselor.

The basic information acquired at step S1010 means initial data on the client, and may be configured of information collected by the system to comprehensively evaluate the psychological state of the client. For example, the basic information may include personal information of the client (e.g., gender, age, education level, etc.), self-report psychological test results, and lifelog data (e.g., daily activities, sleep patterns, etc.).

At step S1020, the apparatus 100 may acquire biometric information of the client.

In order to analyze the psychological state of the client, the biometric information may include physiological information, behavioral information, vocal information, and verbal information of the client collected by the apparatus 100. The apparatus 100 may include various sensors capable of detecting biometric information inside the apparatus 100 to acquire the biometric information, or may acquire biometric information in real time through an external device.

For example, physiological information among the biometric information may include physical responses such as the heart rate, skin conductance, breathing pattern, blood pressure, and eye tracking of the client. The heart rate and blood pressure have a tendency of increasing in a stress or tension situation, and are useful for detecting changes in a psychological state such as lying. The skin conductance changes due to increase in the perspiration, and the breathing pattern may appear in the form of repeating shallow and deep breath or breathing at an irregular rate. In addition, the frequency of blinking eyes as eye tracking shows a characteristic of increasing or decreasing in a tensed state, and is utilized for analyzing psychological changes.

For example, the apparatus 100 may classify signals acquired as biometric signals by eye tracking into variables as shown in Table 1.

TABLE 1

| Variable names | Definitions |
|---|---|
| Fixation duration [ms] | Duration of fixing eyes on a specific location |
| Number of fixations | Frequency and number of times of fixing eyes |
| | on a specific point for at least 100 ms |
| Time to first | Time point of fixing eyes for 100 to 300 ms or |
| fixation | more first (Time point when fixing eyes on |
| | stimulus first) |
| Entry time | Time point of fixing eyes on a specific stimulus |
| | target first after entire stimulus is exposed |
| | (ability of acquiring visual attention to a |
| | specific stimulus) |
| Fixation time | Relative ratio of a numeric value showing |
| ratio [%] | fixation of eyes for 100 ms or more within |
| | 1 degree of viewing angle |
| End time | Time taken to next stimulus after a stimulus |
| | is presented (last time seeing the stimulus) |
| Dwell time [ms] | Total time of dwelling eyes on a specific |
| | area (similar to fixing eyes, but calculated |
| | to include even the saccade) |
| Dwell time ratio [%] | Relative ratio of dwelling eyes on a specific area |
| Saccade count | Movement from one point to another point |
| Saccadic velocity | Velocity of moving eyes from one point to |
| | another point |
| Saccadic amplitude | Distance between points of fixing eyes |
| Pupilometer | Change in pupil size induced by brain activity |
| | (Set to 1 as the largest possible pupil diameter |
| | and to 0 as the smallest possible diameter |
| | when measured) |
| Scanpath | Pattern of eye movement |
| Blink | Number of time of blinking eyes |

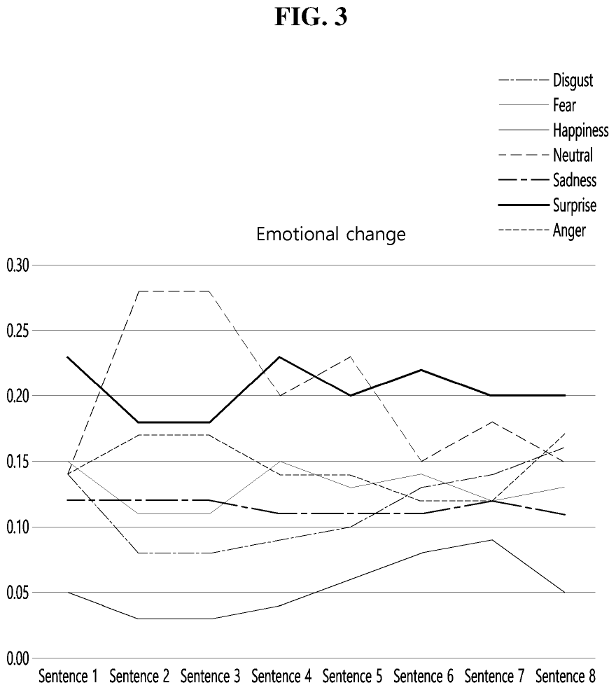

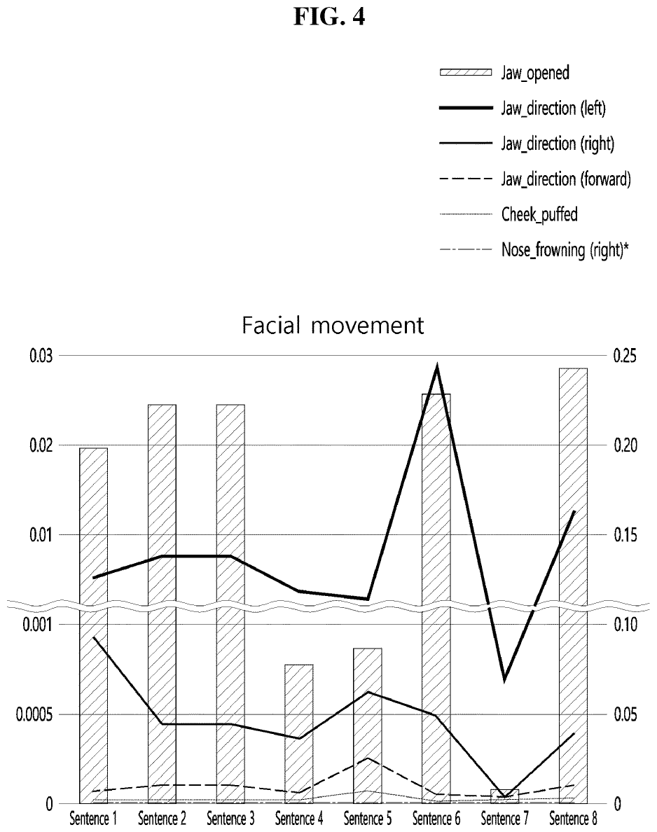

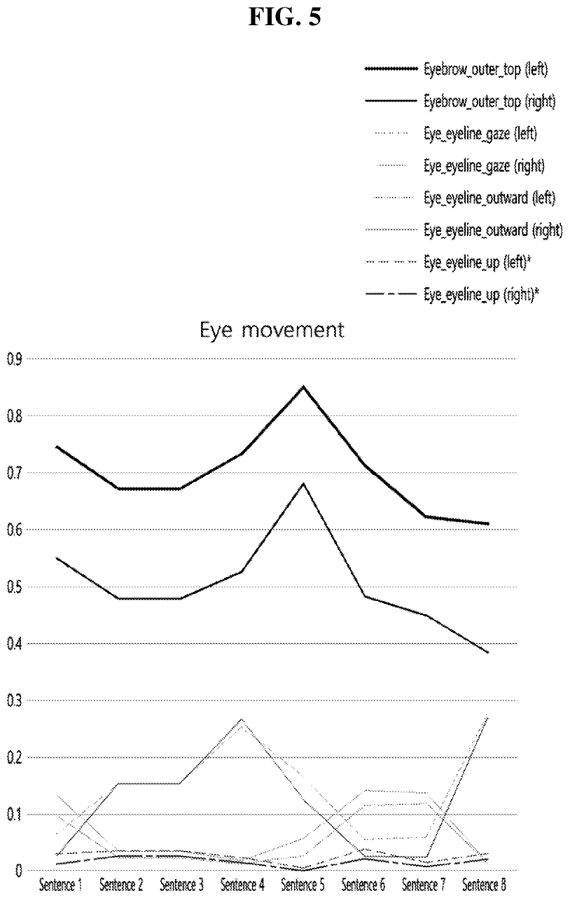

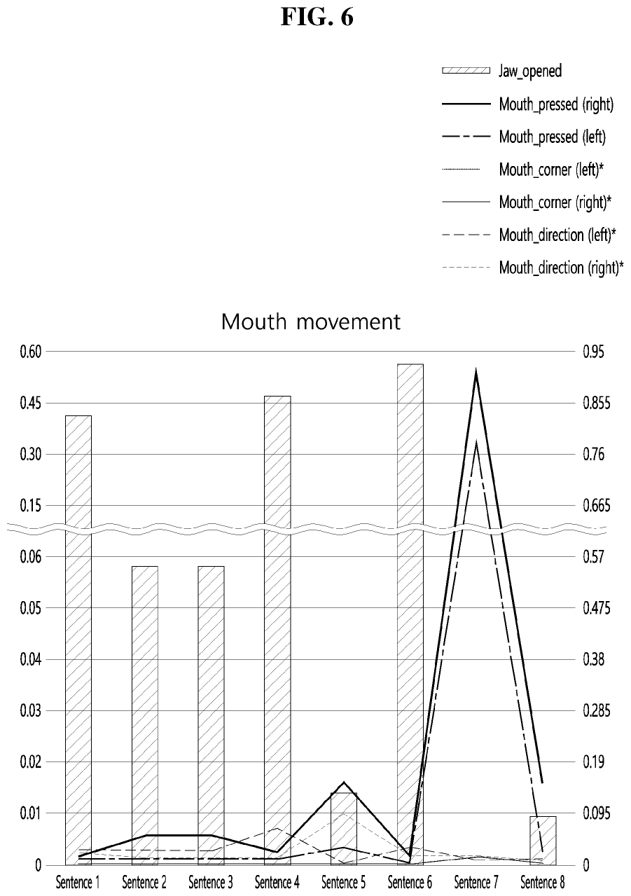

For example, behavioral information, among the biometric data, may include information on the gaze of the client, changes in the facial expression, and changes in the body posture. Examples thereof are described with reference to FIGS. 3 through 6.

FIGS. 3 to 6 are graphs showing values of change measuring the emotional change, facial movement, eye movement, and mouth movement of a client from a specific sentence.

Referring to FIGS. 3 to 6, tendency of rapidly moving the eyes left and right or avoiding eye contact as the gaze of a client may be interpreted as a signal of configuring new information rather than recalling memories. In addition, a subtle change in the facial expression, such as trembling of the corners of the mouth or movement of eyebrows, may reflect a psychological state that the client unconsciously reveals. In addition, a tensioned and uncomfortable posture of the body and a behavior of crossing arms, shaking feet, or twisting body are important indicators for grasping the psychological state of the client.

For example, the apparatus 100 may acquire movement of the gaze of the client or changes in the facial expression from a specific sentence as a biometric signal as shown in Tables 2 and 3.

TABLE 2

| | Sentence | Sentence | Sentence | Sentence | Sentence | Sentence | Sentence | Sentence |
|---|---|---|---|---|---|---|---|---|
| Classifications | 1 | 2 | 3 | 4 | 5 | 6 | 7 | 8 |
| Emotional_hatred | 0.14 | 0.08 | 0.08 | 0.09 | 0.10 | 0.13 | 0.14 | 0.16 |
| Emotional_horror | 0.15 | 0.11 | 0.11 | 0.15 | 0.13 | 0.14 | 0.12 | 0.13 |
| Emotional_happiness | 0.05 | 0.03 | 0.03 | 0.04 | 0.06 | 0.08 | 0.09 | 0.05 |
| Emotional_neutral | 0.14 | 0.28 | 0.28 | 0.20 | 0.23 | 0.15 | 0.18 | 0.15 |
| Emotional_sadness | 0.12 | 0.12 | 0.12 | 0.11 | 0.11 | 0.11 | 0.12 | 0.11 |
| Emotional_surprise | 0.23 | 0.18 | 0.18 | 0.23 | 0.20 | 0.22 | 0.20 | 0.20 |
| Emotional_anger | 0.14 | 0.17 | 0.17 | 0.14 | 0.14 | 0.12 | 0.12 | 0.17 |
| Eyebrow_lower (left) | 0.00 | 0.00 | 0.00 | 0.00 | 0.00 | 0.00 | 0.00 | 0.00 |
| Eyebrow_lower | 0.00 | 0.00 | 0.00 | 0.00 | 0.00 | 0.00 | 0.00 | 0.00 |
| (right) | | | | | | | | |
| Eyebrow_inner_top | 0.67 | 0.66 | 0.66 | 0.73 | 0.87 | 0.71 | 0.61 | 0.59 |
| Eyebrow_outer_top | 0.75 | 0.67 | 0.67 | 0.73 | 0.85 | 0.71 | 0.62 | 0.61 |
| (left) | | | | | | | | |
| Eyebrow_outer_top | 0.55 | 0.48 | 0.48 | 0.53 | 0.68 | 0.48 | 0.45 | 0.38 |
| (right) | | | | | | | | |
| Eye_blink (left) | 0.12 | 0.07 | 0.07 | 0.10 | 0.64 | 0.11 | 0.22 | 0.10 |
| Eye_blink (right) | 0.15 | 0.05 | 0.05 | 0.08 | 0.67 | 0.13 | 0.22 | 0.08 |
| Eye_eyeline_down | 0.37 | 0.28 | 0.28 | 0.40 | 0.80 | 0.29 | 0.54 | 0.32 |
| (left) | | | | | | | | |
| Eye_eyeline_down | 0.35 | 0.27 | 0.27 | 0.37 | 0.79 | 0.29 | 0.48 | 0.30 |
| (right) | | | | | | | | |
| Eye_eyeline_gaze | 0.07 | 0.15 | 0.15 | 0.25 | 0.17 | 0.05 | 0.06 | 0.27 |
| (left) | | | | | | | | |
| Eye_eyeline_gaze | 0.13 | 0.03 | 0.03 | 0.02 | 0.06 | 0.14 | 0.14 | 0.01 |
| (right) | | | | | | | | |
| Eye_eyeline_out (left) | 0.10 | 0.02 | 0.02 | 0.01 | 0.03 | 0.11 | 0.12 | 0.01 |
| Eye_eyeline_out | 0.03 | 0.15 | 0.15 | 0.27 | 0.13 | 0.03 | 0.02 | 0.27 |
| (right) | | | | | | | | |
| Eye_eyeline_up (left) | 0.03 | 0.03 | 0.03 | 0.02 | 0.00 | 0.04 | 0.02 | 0.03 |
| Eye_eyeline_up | 0.01 | 0.03 | 0.03 | 0.01 | 0.00 | 0.02 | 0.01 | 0.02 |
| (right) | | | | | | | | |
| Eye_frowning (left) | 0.34 | 0.27 | 0.27 | 0.26 | 0.57 | 0.37 | 0.41 | 0.30 |
| Eye_frowning (right) | 0.23 | 0.15 | 0.15 | 0.12 | 0.37 | 0.22 | 0.26 | 0.18 |
| Eye_dilated (left) | 0.01 | 0.02 | 0.02 | 0.01 | 0.00 | 0.01 | 0.00 | 0.01 |
| Eye_dilated (right) | 0.00 | 0.01 | 0.01 | 0.01 | 0.00 | 0.01 | 0.00 | 0.01 |
| Cheek_puffed | 0.00 | 0.00 | 0.00 | 0.00 | 0.00 | 0.00 | 0.00 | 0.00 |
| Cheek_frowning (left) | 0.00 | 0.00 | 0.00 | 0.00 | 0.00 | 0.00 | 0.00 | 0.00 |
| Cheek_frowning | 0.00 | 0.00 | 0.00 | 0.00 | 0.00 | 0.00 | 0.00 | 0.00 |
| (right) | | | | | | | | |
| Chin_direction | 0.00 | 0.00 | 0.00 | 0.00 | 0.00 | 0.00 | 0.00 | 0.00 |
| (forward) | | | | | | | | |
| Chin_direction (left) | 0.01 | 0.01 | 0.01 | 0.01 | 0.00 | 0.02 | 0.00 | 0.01 |

TABLE 3

| Jaw_opened | 0.20 | 0.22 | 0.22 | 0.08 | 0.09 | 0.23 | 0.01 | 0.24 |
|---|---|---|---|---|---|---|---|---|
| Jaw_direction (right) | 0.00 | 0.00 | 0.00 | 0.00 | 0.00 | 0.00 | 0.00 | 0.00 |
| Nose_frowning (left) | 0.00 | 0.00 | 0.00 | 0.00 | 0.00 | 0.00 | 0.00 | 0.00 |
| Nose_frowning (right) | 0.00 | 0.00 | 0.00 | 0.00 | 0.00 | 0.00 | 0.00 | 0.00 |
| Mouth_closed | 0.02 | 0.02 | 0.02 | 0.01 | 0.01 | 0.02 | 0.00 | 0.01 |
| Mouth_dimple (left) | 0.00 | 0.00 | 0.00 | 0.00 | 0.00 | 0.00 | 0.01 | 0.00 |
| Mouth_dimple (right) | 0.00 | 0.00 | 0.00 | 0.00 | 0.01 | 0.00 | 0.05 | 0.00 |
| Mouth_corner (left) | 0.00 | 0.00 | 0.00 | 0.00 | 0.00 | 0.00 | 0.00 | 0.00 |
| Mouth_corner (right) | 0.00 | 0.00 | 0.00 | 0.00 | 0.00 | 0.00 | 0.00 | 0.00 |
| Mouth_narrowed | 0.06 | 0.05 | 0.05 | 0.04 | 0.02 | 0.06 | 0.00 | 0.01 |
| Mouth_direction (left) | 0.00 | 0.00 | 0.00 | 0.01 | 0.00 | 0.00 | 0.00 | 0.00 |
| Mouth_lowered (left) | 0.00 | 0.00 | 0.00 | 0.00 | 0.01 | 0.00 | 0.00 | 0.01 |
| Mouth_lowered (right) | 0.00 | 0.00 | 0.00 | 0.00 | 0.01 | 0.00 | 0.00 | 0.01 |
| Mouth_pressed (left) | 0.00 | 0.00 | 0.00 | 0.00 | 0.00 | 0.00 | 0.33 | 0.0 |
| Mouth_pressed (right) | 0.00 | 0.01 | 0.01 | 0.00 | 0.02 | 0.00 | 0.51 | 0.02 |
| Mouth_pursed | 0.83 | 0.55 | 0.55 | 0.87 | 0.13 | 0.93 | 0.00 | 0.09 |
| Mouth_direction (right) | 0.00 | 0.00 | 0.00 | 0.00 | 0.01 | 0.00 | 0.00 | 0.00 |
| Mouth_rolled (down) | 0.02 | 0.01 | 0.01 | 0.02 | 0.01 | 0.03 | 0.04 | 0.02 |
| Mouth_rolled (up) | 0.04 | 0.03 | 0.03 | 0.04 | 0.01 | 0.05 | 0.02 | 0.02 |
| Mouth_shrinked (down) | 0.00 | 0.00 | 0.00 | 0.00 | 0.00 | 0.00 | 0.18 | 0.00 |
| Mouth_shrinked (up) | 0.00 | 0.00 | 0.00 | 0.00 | 0.01 | 0.00 | 0.01 | 0.01 |
| Mouth_smiling (left) | 0.00 | 0.00 | 0.00 | 0.00 | 0.00 | 0.00 | 0.02 | 0.00 |
| Mouth_smiling (right) | 0.00 | 0.00 | 0.00 | 0.00 | 0.00 | 0.00 | 0.02 | 0.00 |
| Mouth_opened (left) | 0.00 | 0.00 | 0.00 | 0.00 | 0.00 | 0.00 | 0.01 | 0.00 |
| Mouth_opened (right) | 0.00 | 0.00 | 0.00 | 0.00 | 0.00 | 0.00 | 0.00 | 0.00 |
| Mouth_raised (left) | 0.00 | 0.00 | 0.00 | 0.00 | 0.00 | 0.00 | 0.00 | 0.00 |
| Mouth_raised (left) | 0.00 | 0.00 | 0.00 | 0.00 | 0.00 | 0.00 | 0.00 | 0.00 |

TABLE 4

| | Sentence | Sentence | Sentence | Sentence | Sentence | Sentence | Sentence | Sentence |
|---|---|---|---|---|---|---|---|---|
| | 1 | 2 | 3 | 4 | 5 | 6 | 7 | 8 |
| Emotional | Good | Comfortable | Busy | Neutral | Comfortable | Precious | Sick | Anxious |
| words | | | | | | | | |

For example, vocal information, among the biometric data, may include information on the pitch of voice, speed of speech, change in intonation, and trembling of voice. For example, when feeling nervous or stressed, the voice may be higher or lower than usual, and the speed of speech may increase or decrease. In addition, intonation may also change to be different from the usual, and the way of raising or lowering the end of a sentence may vary. These vocal characteristics provide important clues for analyzing the psychological state of a client.

For example, verbal information, among the biometric information, may include information obtained by quantifying the emotional value, emotional intensity, prosociality, and personality of a client with respect to the response of the client to a given question.

The emotional value is a numerical value obtained by identifying the emotion within a given text and determining whether the attitude of the respondent is positive, neutral, or negative. For example, when the emotional value is a value between 1 and −1, an emotional value closer to 1 may be interpreted as positive, an emotional value closer to −1 as negative, and an emotional value closer to 0 as neutral. The apparatus 100 may calculate the emotional value of the entire sentence according to the context after assigning a positive or negative score on the basis of a word dictionary in which positive or negative characteristics are predefined for calculation of the emotional value. For example, although “I am happy” is evaluated as positive, “I am not happy” is evaluated as negative considering the influence of negative words. In addition, “I am very sad” may be evaluated as more negative than “I am sad” due to the influence of the modifier.

The emotional intensity represents the intensity of an emotional expression, and is a numerical value obtained by quantifying the density of using emotional words within a given text. The higher the emotional intensity value, the ratio of using emotional words may be interpreted as higher. When negative and positive words are used at similar ratios, the emotional value is evaluated to be close to 0, but the emotional intensity may be evaluated to be higher than that of a text without including emotional words.

The prosociality is a numerical value obtained by quantifying the degree of using prosocial words within a given text. The apparatus 100 may define a list of prosocial words in advance and use the list to calculate a prosociality score. For example, the apparatus 100 may classify prosocial words into two levels, including top-level prosocial core words (Level 1) reflecting positive characters such as “honest” and “open”, and words of next highest level prosocial character (Level 2). At this point, the apparatus 100 may calculate a prosociality score on the basis of the frequency of prosocial words appearing in a text, i.e., calculates a comprehensive prosociality score by assigning a higher weight to words of Level 1.

The personality is a numerical value obtained by quantifying the degree of using less frequently appeared words. The apparatus 100 may classify words into four levels and use them to evaluate personality. For example, on the basis of frequency of words appearing in the response data of 1,000 people, Level 1 may be defined as a case where a word appears in more than ⅓ of respondents, Level 2 as a case where a word appears in more than 5% of respondents, Level 3 as a case where a word appears in more than 2% of respondents, and Level 4 as a case where a word appears in less than 2% of respondents. At this point, the lower the frequency of occurrence of a word, the apparatus 100 gives a higher score, and the closer the word is to Level 4, a comprehensive personality score may be calculated by assigning a higher weight.

The apparatus 100 may analyze the response of the client and perform a next process to quantify the emotional value, emotional intensity, prosociality, and personality of the response of the client. First, the apparatus 100 may separate the text corresponding to the response in units of morphemes (tokenize) for text preprocessing of the response, and then classify the part of speech, dependency, and negative expression of words to analyze contextual meaning thereof. Subsequently, the apparatus 100 may calculate the frequency of appearance of all words used in the response to calculate frequency of words. Accordingly, the apparatus 100 calculates scores of the emotional value, emotional intensity, prosociality, and personality, and calculates a percentile score by utilizing the mean and standard deviation of the normative data (data of 1,000 people) for all scores (emotional value, emotional intensity, prosociality, and personality) to grasp the position of the score of each area on the basis of the normative data.

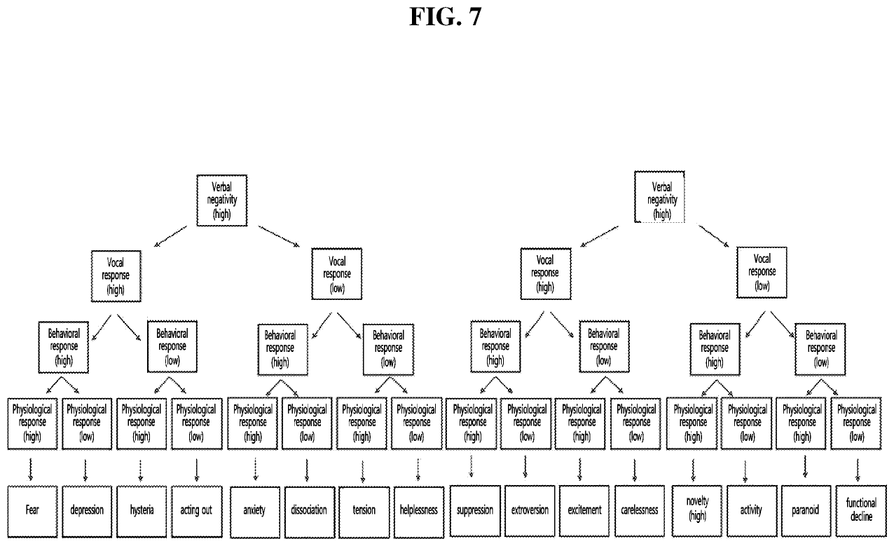

FIG. 7 is an exemplary view showing an operation of classifying the percentile of a value derived from a biometric signal of a client into an upper or lower level according to an embodiment.

Referring to FIG. 7, the apparatus 100 may classify a response of the client into an upper or lower level based on percentiles on the basis of the percentile of a value derived from any one among the emotional value, emotional intensity, prosociality, and personality of the emotional response of the client, and configure a decision tree for diagnosing the psychological state of the client. For example, when verbal negativity is expressed as the highest percentile, it may be evaluated as “fear”, and when verbal negativity is expressed as the lowest percentile, it may be evaluated as “functional decline”. The psychological state classified as described may be mapped to a biometric signal and utilized as an input of the LMM model described below.

In addition, at step S1020, the apparatus 100 classifies numerical or classification values of physiological information, behavioral information, vocal information, and verbal information corresponding to biometric signals of the client on the basis of the criteria predefined as shown in Tables 5 to 7, and may classify a biometric signal corresponding to any one item of Tables 5 to 7 as a specific psychological factor. The apparatus 100 may store the classified psychological factor to be mapped to the acquired biometric signal. At this point, the psychological factor mapped to the biometric signal may also be utilized as an input of the LMM model described below. All the evaluation items shown in Tables 5 to 7 are generated on the basis of percentile information after standardizing the data on the basis of the normative data of 1,000 people. At this point, the measured psychological factors include types classified as depression, fear, stress, pain, discomfort, helplessness, passivity, passive-aggressiveness, reaction formation, hypomania, anxiety, tension, attention, aggression, resistance, rigidity, social desirability, suppression, denial, dissociation, excitement, carelessness, novelty seeking, extroversion, activity, paranoid, functional decline, acting out, and thought disorder.

TABLE 5

| | | | Measured | |
|---|---|---|---|---|
| Response | Measurement | | psychological | Examples of |
| classifications | items | Definitions | factors | situations |
| Biomedical | Fixation | Duration of fixing | Attention, | Pay attention to a |
| information | duration | eyes on a specific | Concentration | specific area |
| Eye tracking | | location target | | |
| Biomedical | Number of | Number of times of | Attention, | See a specific area |
| information | fixations | shifting eyes to a | Interest | several times |
| Eye tracking | | specific target | | |
| Biomedical | Time to first | Time of fixing eyes at | Attention | Time of gazing at a |
| information | fixation | a specific target first | | specific target first |
| Eye tracking | | | | |
| Biomedical | Entry time | Time required for eyes | Attention | Time required for eyes |
| information | | to enter a specific area | | to arrive at a specific |
| Eye tracking | | | | area |
| Biomedical | Fixation time | Ratio of duration of | Concentration, | Analyze ratio of |
| information | ratio | fixing eyes to total | Distraction | gazing at a specific |
| Eye tracking | | time | | area out of the entire |
| | | | | area |
| Biomedical | End time | Time of leaving eyes | Attention | Analyze time of |
| information | | from a specific target | | leaving eyes from a |
| Eye tracking | | | | specific area |
| Biomedical | Dwell time | Duration of dwelling | Strong interest | Case of fixing eyes at |
| information | | eyes on a specific | | a specific area for a |
| Eye tracking | | target | | long time |
| Biomedical | Dwell time | Ratio of fixing eyes | Attention, | Compute ratio of |
| information | ratio | on a specific target to | Concentration | fixing eyes at a |
| Eye tracking | | total time of seeing | | specific area |
| Biomedical | Saccade | Number of times of | Search, | Shift eyes from one |
| information | count | shifting eyes to | Anxiety, | area to another area |
| Eye tracking | | another target | Tension, | while searching, Shift |
| | | | Avoidance | eyes for avoidance |
| Biomedical | Saccadic | Average speed of | Efficiency of | Rapidly shift eyes |
| information | velocity | shifting eyes | information | during information |
| Eye tracking | | | search | search |
| Biomedical | Saccadic | Average distance | Concentration | Distance of shifting |
| information | amplitude | between eye shifts | of eye shift | eyes comparing two |
| Eye tracking | | | | targets spaced apart |
| Biomedical | Diameter of | Change in the size of | Tension, | Dilation of pupils |
| information | pupil | pupils | Interest | when seeing an |
| Eye tracking | | | | interesting target |
| Biomedical | Eye shift | Direction and pattern | Search, | Rapidly gazing at a |
| information | | of eyes | Confusion | specific point on the |
| Eye tracking | | | | map |

TABLE 6

| | | | | When one does not |
|---|---|---|---|---|
| Biometric | | | | believe someone's |
| information - | | Behavior of | Doubt, | speaking, feel |
| eyes | Eyes_frowning | squinting | Discomfort | uncomfortable |
| Biometric | Eyes_wide open | Behavior of | Surprise, Fear | When telling shocking |
| information - | | opening eyes | | story, when hearing |
| eyes | | wide | | interesting story |
| Biometric | Pupil dilation | Degree of pupil | Interest, Tension | Pupils dilate in |
| information - | | dilation | | emotionally agitated |
| eyes | | | | state |
| Biometric | Cheek_puffed | Behavior of | Confidence, | Express confidence by |
| information - | | puffing cheeks | Exaggerated | puffing cheeks |
| face | | | expression | |
| Biometric | Cheek_frowning | Motion of | Doubt, | Frown cheeks in |
| information - | | frowning cheeks | Discomfort | uncomfortable |
| face | | | | conversation |
| Biometric | Chin_direction | Measure | Determination, | Fix chin while making |
| information - | | movement of | Tension | decision |
| face | | chin | | |
| Biometric | Chin_direction | Behavior of | Concentration, | Push chin forward |
| information - | | pushing chin | Will | expressing strong will |
| face | | forward | | |
| Biometric | Jaw_opened | Behavior of | Surprise, | State of being |
| information - | | opening mouth | Intention of | surprised or before |
| face | | | speaking | speaking |
| Biometric | Mouth_direction | Maintain lips | Neutral state | State of not speaking |
| information - | | basically | | to keep neutral |
| face | | | | position |
| Biometric | Mouth_closed | Behavior of | Silence, | Close lips and keep |
| information - | | closing mouth | Resistance, | silent |
| face | | | Oppression | |
| Biometric | Mouth_dimple | Dimples shown | Satisfaction, | Dimples are shown in |
| information - | | on both sides of | Pride | satisfied state |
| face | | lips | | |
| Biometric | Mouth_corners | Behavior of | Sadness, | Mouth corners turn |
| information - | | turning mouth | Disappointment | down in sadness |
| face | | corners down | | |
| Biometric | Mouth_narrowed | Motion of | Doubt, Wariness | Narrow lips with |
| information - | | narrowing mouth | | caution |
| face | | | | |
| Biometric | Mouth_lowered | Motion of turning | Disappointment, | Lower lip turns down |
| information - | | lower lip down | Resignation | in disappointment |
| face | | | | |

TABLE 7

| | | | | Maintain oxygen |
|---|---|---|---|---|
| Biometric | Oxygen | Ratio of oxygen | | saturation in healthy |
| information | saturation | saturation in blood | Healthy state | state |
| Biometric | Change in | Change in facial | Expression of | Express emotion by |
| information | facial | expression due to | emotion | laughter |
| | expression | change in facial | | |
| | | muscles | | |
| Behavioral | Change in | Change in posture of | Discomfort, | Change posture in |
| information | body posture | whole body | Tension | tensed state |
| Behavioral | Body | Frequency and pattern | Active | Keep moving body in |
| information | movement | of body movement | | anxiety |
| Voice | Pitch of voice | Changes in tone and | Emphasis, | Pitch of voice goes up |
| information | | pitch of voice | Expression of | when exited |
| | | | emotion | |
| Voice | Speed of | Pace and speed of | Tension, | Speak faster when |
| information | speech | speech | Expression of | nervous |
| | | | emotion | |
| Voice | Intonation of | Pattern of intonation | Emphasis, | Intonation changes |
| information | speech | of speech | Emotional | when emphasizing |
| | | | state | |
| Voice | Tremor of | Tremors revealed in | Tension, | Voice trembles with |
| information | voice | voice | Anxiety | tension |
| Voice | Voice | Information included | Information | Transfer positive |
| information | information | in voice | transfer | information on |
| | | | | specific subject |
| Language | Analysis of | Subject and content | Interest in | Express interest in |
| information | speech | revealed in speech | subject | subject |
| Language | Pattern of | Pattern of words | Thinking | Show interest through |
| information | words used | repeatedly used in | pattern | repeated words |
| | | speech | | |
| Language | Complexity | Structural complexity | Cognitive | Reveal thinking using |
| information | of sentence | of sentence | ability | long sentence |
| Language | Frequency of | Frequency of using | Usage habit | Frequently use |
| information | words | specific words | | specific words |
| Language | Preferred | Most preferred subject | Preference, | Repeat easy subjects |
| information | subject | | Avoidance | to avoid frequently |
| | | | | discussed subject and |
| | | | | uncomfortable |
| | | | | conversation |
| Language | Rejected | Subject showing | Rejection, | Subject desired to |
| information | subject | rejection | Discomfort, | avoid |
| | | | Defensiveness | |
| Language | Key subject | Important subject in | Key point of | Key point of |
| information | | conversation | discussion | conversation |

In addition, in order to evaluate the stress level and defensive attitude of the client, at step S1020, the apparatus 100 may determine inconsistency between at least two pieces of information, among the physiological information, behavioral information, vocal information, and verbal information, corresponding to the biometric signal. At this point, when the determined inconsistency exceeds a preset threshold, the apparatus 100 may determine that the acquired biometric signal is a psychological resistance or false report of the client, and reacquire the biometric signal of the client.

For example, when a client, who uses verbal information of a relatively high positive value on the basis of standard deviation unit in the standardized normative data, shows anxious or avoidant eye movements in the facial expression, voice, and gaze that are relatively less positive compared to the verbal information, the apparatus 100 may evaluate that the client has a defensive tendency. In addition, although the client says something that does not have any particular abnormal opinion in the content of verbal conversation in the process of an interview, when the client does not stably maintain the gaze on a target stimulus in the gaze among the biometric signal, the client may be diagnosed as having an attention problem. In addition, in the case of a client who lies, a high-level deviation may be shown in the verbal message from the aspect of balance between the evaluation items of the emotional value, emotional intensity, prosociality, and personality in the normative data, and the ratio of discrepancy between the verbal message and various biometric information may be high. In addition, in the case of a client who has a defensive tendency, although the level of response related to a negative emotional reaction in a verbal response is low on the basis percentile and the level of a response related to prosociality is high, in the nonverbal information and biometric indicators, a high score may be shown in the biometric indicators indicating a negative response or emotional instability.

At step S1030, the apparatus 100 may generate evaluation information about the psychological state of the client by utilizing the LMM model on the basis of the acquired basic information and biometric information.

The Large Multimodal Model (LMM) is a large-scale neural network model that comprehensively processes and analyzes diverse modal data such as text, voice, video, biometric signals, and the like. For example, the LMM model may be designed on the basis of a transformer architecture, and multimodal data may be processed efficiently and precisely through the LMM model.

The LMM model based on the transformer may generate vector information combining the basic information and the biometric information by utilizing a self-attention mechanism or a cross-attention mechanism, and generate evaluation information about the psychological state of the client on the basis of the combined vector information. The self-attention mechanism puts emphasis on important features in the modal data, and the cross-attention mechanism may grasp interactions between different modal data and generate combined vector information. The combined vector information is used to comprehensively evaluate the psychological state of the client.

For example, the apparatus 100 may generate evaluation information about the psychological state of the client on the basis of the self-attention mechanism between text specifying the basic information and text defining the determined psychological factor. In this process, the self-attention mechanism analyzes important keywords and contextual correlations in the text data and helps in grasping the psychological characteristics of the client.

For example, the apparatus 100 may generate evaluation information about the psychological state of the client on the basis of the cross-attention mechanism between text specifying the basic information and an image or a video specifying the biometric information. In this process, the cross-attention mechanism may derive complex information related to the psychological state of the client by analyzing the correlation between text data and image data, and generate precise evaluation information on the basis of the complex information.

In this way, the apparatus 100 may efficiently process multi-modal data and evaluate the psychological state of the client in various ways by utilizing the self-attention mechanism and the cross-attention mechanism.

At step S1040, the apparatus 100 may provide feedback information to the client or the counselor counseling the client on the basis of the evaluation information.

For example, the feedback information may include temporal tendency in the change of the psychological state of the client and a warning about a psychologically high-risk group. The temporal tendency information indicates how the psychological state of the client has changed over a specific period of time, and allows both the client and the counselor to grasp whether the psychological stability is improved or will be deteriorated. The warning about a psychologically high-risk group induces appropriate measures by informing a situation in which the client is psychologically unstable or an immediate intervention is required.

In addition, the feedback information may provide a result of specific analysis on the emotional state and stress level of the client. For example, the apparatus 100 may identify major stress factors of the client and provide customized advice or behavioral guidance to alleviate the stress. The feedback information provided to the counselor may include detailed data needed to clearly understand the current state of the client and design counseling strategies.

In addition, the apparatus 100 may reflect a result of analyzing verbal and nonverbal data generated from the real-time conversations between the client and the counselor into the feedback to emphasize key factors that should be considered in the counseling process. Through this, the client may understand his or her psychological state further better, and the counselor may provide more effective and personalized psychological support.

At step S1050, the apparatus 100 may acquire an additional input from the counselor, and generate information on the psychological counseling or treatment plan by inputting a command corresponding to the additional input and evaluation information into the LMM model.

Here, the additional input of the counselor may include data for understanding the state of the client more deeply or specifying the direction of counseling.

For example, the counselor may provide the current psychological state, details of previous counseling, and a result of observing a specific behavior or response as an additional input. The additional input is input into the LMM model and processed to be integrated with existing basic information and biometric information.

In addition, the command corresponding to the additional input means a command text that instructs the LMM model to perform a specific task or process on the basis of the additional input of the counselor.

For example, information on the generated treatment plan may include configuration of a customized counseling session, a proposal of stress management method, a behavior modification plan in each step, and the like for improving the psychological stability of the client.

As described above, the apparatus 100 may analyze the additional input provided to the counselor through the LMM model, and propose a counseling strategy adjusted in real time or recommend a detailed treatment plan targeting a specific psychological state of the client.

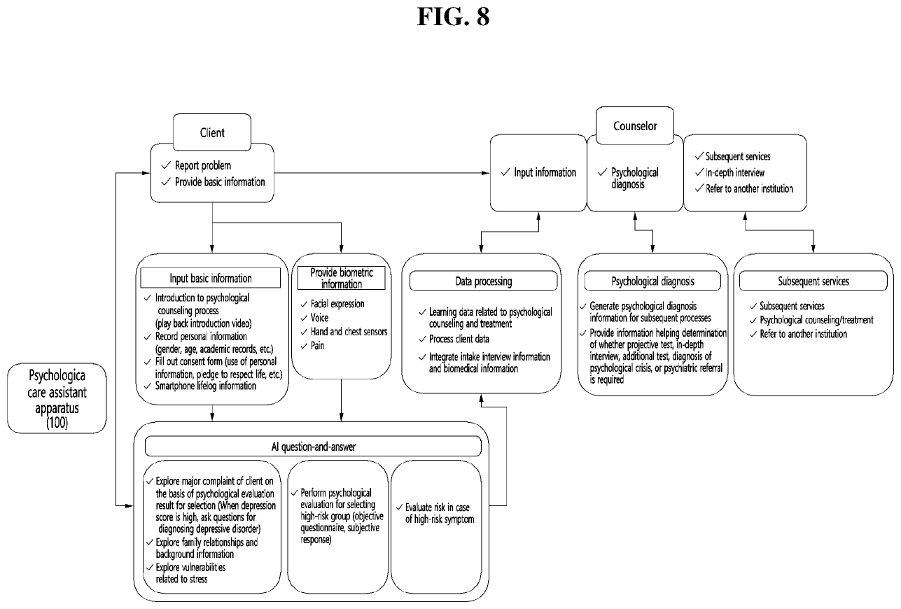

Hereinafter, scenarios of applying the operation of the apparatus 100 described above in various situations are described with reference to FIGS. 8 to 12. In FIGS. 8 to 12, it is described assuming that the LMM model utilized by the apparatus 100 according to an embodiment of the present invention is implemented in the form of a psychological care assistant. Here, the psychological care assistant may include a digital human that evaluates the psychological state of a client through interactions with the client and supports counseling and treatment. In addition, the psychological care assistant may include a digital human that assists the counseling session in cooperation with the counselor. That is, the psychological care assistant provides the counselor with a result of analyzing the psychological state of the client and real-time feedback, and may induce self-expressions from the client through customized questions or maximize effectiveness of the counseling process.

FIG. 8 is a view showing the concept of interactions between the apparatus 100 and a client and a counselor in an initial intake interview situation in the psychological counseling and treatment field according to an embodiment.

Referring to FIG. 8, the apparatus 100 allows a client to have a preliminary interview through the LMM model-based psychological care assistant, and may arrange the client to have psychological counseling and treatment of an expert when necessary. In addition, the apparatus 100 helps in understanding the client broadly on the basis of various psychological state analysis data including biometric information and the like, and the analysis data facilitates deeper understanding and therapeutic effects in the counseling with an expert.

To this end, in order for the apparatus 100 to collect basic information, the client may provide the basic information through an initial intake interview of 20 to 30 minutes as shown in FIG. 8. At this point, the psychological counseling process may be introduced to the client, and collection of personal information (e.g., gender, age, academic records, etc.), completion of a consent form for using personal information, provision of smartphone lifelog information, and the like may be performed.

Subsequently, in order to collect the biometric information, the apparatus 100 may comprehensively evaluate biometric information of the client by applying techniques of recognizing psychological pain and detecting false reports. At this point, the apparatus 100 may comprehensively process various biometric information and psychological test information, including facial expression information, micro-movements of eyebrows, lips, eye blinking, eye tracking, pupil dilation, and the like, movements of face, neck, upper and lower body, and the like, verbal and nonverbal information related to voice, acoustic information, body tension, pain-related indicators, blood pressure, heart rate, respiration, and the like, through the LMM model, and utilize the information for subsequent diagnosis.

At this point, the apparatus 100 may integrate eight types of emotional information, voice recording (text), and eight types of movement information, and present them in the form of a transcript to be used. The collected information may be utilized for classification and diagnosis of masked depression, hysteria, and the like. In addition, the apparatus 100 may utilize the biometric information to compare neutral conversation with conversations related to lies and confirm that the client is lying on a specific topic.

In order to process data, the apparatus 100 may process the collected basic information and biometric information through the LMM model-based psychological care assistant. At this point, the apparatus 100 may process data on the client and generate psychological diagnosis information by utilizing learning data related to psychological counseling and treatment.

The apparatus 100 may select a high-risk group and perform psychological evaluation through question-and-answer with a client on the basis of data processed for the question-and-answer of the LMM-based psychological care assistant, explore major problems of the client on the basis of the result of the psychological evaluation selected through objective questionnaires and subjective responses, and additionally ask questions for diagnosing depressive disorder when a high-risk factor such as depression is found.

In addition, after question-and-answer with the psychological care assistant, the apparatus 100 may perform a projective test, in-depth interview, and additional test according to the state of the client to diagnose subsequent processes, and perform psychological counseling and treatment or refer the client to another institution (counseling center, psychiatry, etc.).

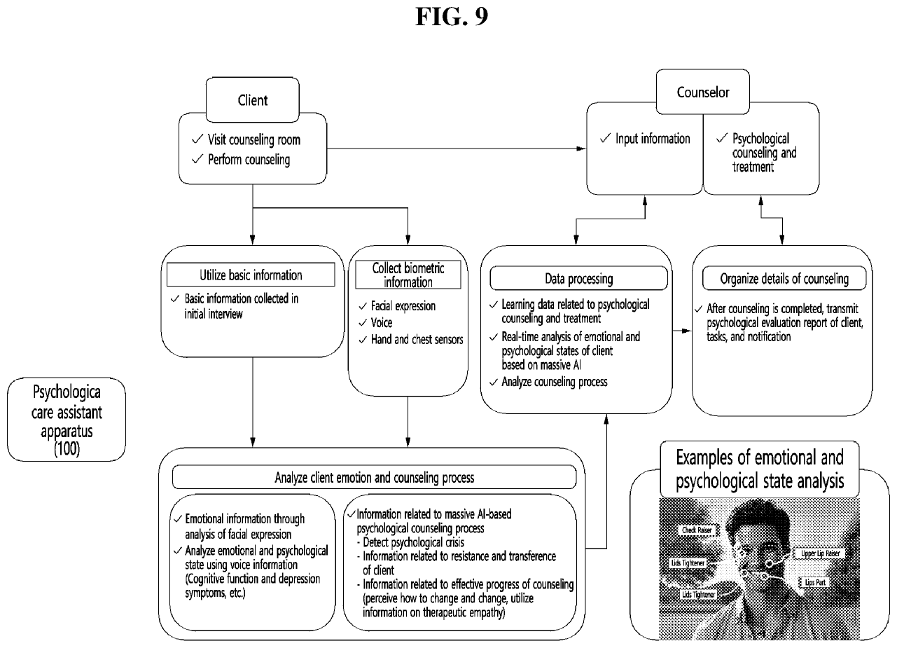

FIG. 9 is a view showing the concept of interactions between the apparatus 100 and a client and a counselor in a situation of assisting a therapeutic expert of individual and group psychological counseling (referred to as a “counselor” in the specification) according to an embodiment.

Referring to FIG. 9, the apparatus 100 allows the counselor to perform counseling by utilizing an LMM-based psychological care assistant, and the counselor may be provided with preliminary information on the client using the apparatus 100 and make a diagnosis on the basis of the psychological state of the client analyzed in real time. At this point, analysis of the psychological state through the LMM-based psychological care assistant is conducted by comprehensively utilizing biometric information and psychological tests. In this process, hindrance that appears through the resistance of the client, such as the masked depression, falsehood, or the like, may be selected through the information related to pain and false behaviors. The counselor may provide more accurate and effective counseling services on the basis of the current state, self-report data, and the like of the client, and various learning data reflected in the database.

To this end, in order to collect the basic information, the apparatus 100 may acquire, as shown in FIG. 9, an initial diagnostic report on the basis of self-report and prior psychological state information as basic information collected through the initial intake interview of the client.

Subsequently, in order to collect the biometric information, the apparatus 100 may collect biometric information for detecting facial expressions, voice, and sensation of the hands and chest of the client. Through this, the current psychological state of the client (e.g., tension, anxiety, anger, etc.) may be grasped more accurately. In addition, the apparatus 100 may analyze facial expressions and pains using a camera and a facial recognition solution, and analyze voice states (e.g., trembling, etc.) using a voice recognition solution.

In order to process data, the apparatus 100 may process the collected basic information and biometric information using the LMM model-based psychological care assistant. At this point, the apparatus 100 may process data on the client and generate psychological diagnosis information by utilizing learning data related to psychological counseling and treatment. During the data analysis, the apparatus 100 may comprehensively analyze explicit and implicit expressions and provide accurate information on the psychological state of the client that is not reflected in the explicit expressions.

For example, the apparatus 100 may grasp changes in the facial expressions, movements, and voice by comparing information between sessions or within a session, and generate information useful for performing the counseling, such as the degree of progress toward the goal of counseling (e.g., alleviation of depression), defense level, rapport level, transference level, or the like, on the basis of the changes.

For example, the apparatus 100 may generate information on high-frequency words representing the major complaint and interest area of the client and low-frequency or missing words reflecting suppression, avoidance, or individual psychological characteristics by performing analysis of word frequency on the basis of verbal information of the client.

For example, the apparatus 100 may explore topics that the client prefers to handle, topics that show rejective reactions, and core issues associated with emotional signals by comprehensively analyzing verbal and nonverbal information. For example, the preferred topics may be topics frequently repeated and frequently combined with positive verbal expressions and nonverbal signals indicating comfort, the rejective topics may be topics abruptly switching to another topic or frequently combined with negative verbal expressions and nonverbal signals indicating emotional tension, and the core issues may be topics associated with signals of emotional escalation, defensiveness, and suppression, topics inducing maladaptive coping patterns of the client, or topics activated by the psychological schema of the client.

For example, when there is a difference between an explicit expression and an implicit expression, the apparatus 100 may provide information on the interpretation of an area where the client is defensive or suppressed or lacking in insight although the client does not express outwardly, or an area where the client complains difficulties excessively compared to psychological pain.

For example, by deeply analyzing voice information, the apparatus 100 may provide information on the problems such as cognitive decline and depression symptoms of the client that are difficult to grasp through an explicit expression.

For example, the apparatus 100 may provide guidance for the diagnosis of the client as details of the counseling are reflected in the system in real time. The apparatus 100 allows the counselor to detect important emotional, cognitive, and behavioral changes of the client in real time, and appropriately respond thereto.

The apparatus 100 may organize details of the counseling on the basis of the report provided by the system after the counseling is completed, and provide reference materials to determine whether additional psychological counseling and treatment are required.

For example, the apparatus 100 may provide the client with key topics to be handled in the subsequent sessions and a list of useful tasks to accomplish the goal of counseling.

For example, the apparatus 100 stores data on the counseling process in the form of a transcript integrating emotional and various psychological diagnosis information to be conveniently utilized for self-monitoring and supervision of the counselor. In addition, although the counselor may utilize only voice information when organizing the transcript in conventional face-to-face counseling, the apparatus 100 may provide the counselor with psychological diagnosis information obtained by comprehensively analyzing multimodal data.

For example, when a psychological crisis of the client is detected, the apparatus 100 may suggest necessary additional measures, such as an emergency interview, psychiatric referral, or the like.

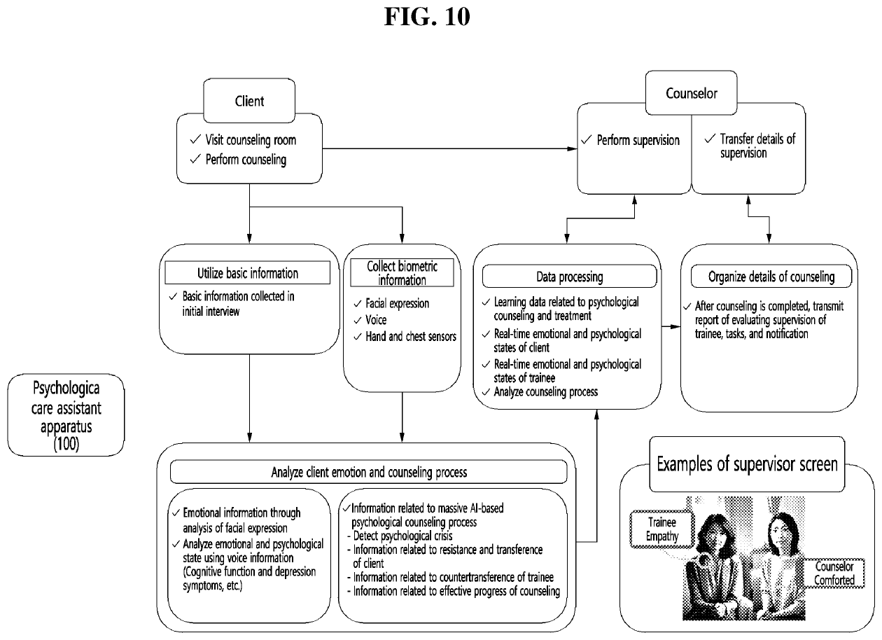

FIG. 10 is a view showing the concept of interactions between the apparatus 100 and a client and a counselor in a situation of assisting a therapeutic supervisor of individual and group psychological counseling (referred to as a “counselor” in the specification) according to an embodiment.

Referring to FIG. 10, the apparatus 100 may perform supervision targeting the psychological counseling and treatment trainees by utilizing an LMM-based psychological care assistant. At this point, provision of information may be performed by comprehensively utilizing biometric information and psychological tests, like in the case where the LMM-based psychological care assistant is utilized in an initial intake interview tool or psychological counseling and treatment. The apparatus 100 may be provided with information based on client-related information and trainee-related information when conducting the psychological counseling and treatment, and perform supervision on the basis of information related to interactions between the client and the psychological counseling and treatment trainees, and analyzed in real time.

In addition, the apparatus 100 may utilize the LMM-based psychological care assistant to perform supervision on the basis of actual empirical evidence on the phenomena of transference and countertransference that appear in the process of psychological counseling and treatment by comprehensively analyzing information related to interactions between the client and the psychological counseling and treatment trainees. Here, transference means that the client re-experiences various emotions about the counselor on the basis of relationships with persons in the past, and countertransference means that the counselor re-experiences various emotions about the client on the basis of relationships with persons in the past.

To this end, the apparatus 100 may acquire initial intake interview information of the client and basic information of the trainees.

In addition, the apparatus 100 may grasp the current psychological state of the client (e.g., tension, anxiety, anger, etc.) more accurately by collecting biometric information for detecting facial expressions, voice, and sensation of the hands and chest of the client. In addition, the apparatus 100 may analyze facial expressions and pains using a camera and a facial recognition solution, and analyze voice states (e.g., trembling, etc.) using a voice recognition solution.

The apparatus 100 may process the collected basic information and biometric information using an LMM-based psychological care assistant. The apparatus 100 may process data on the client and generate psychological diagnosis information by utilizing learning data related to psychological counseling and treatment.

The apparatus 100 may organize details of supervision performed by the supervisor on the basis of the report provided by the LMM model to organize details of the counseling, and provide reference materials to determine whether additional intervention or education is required.

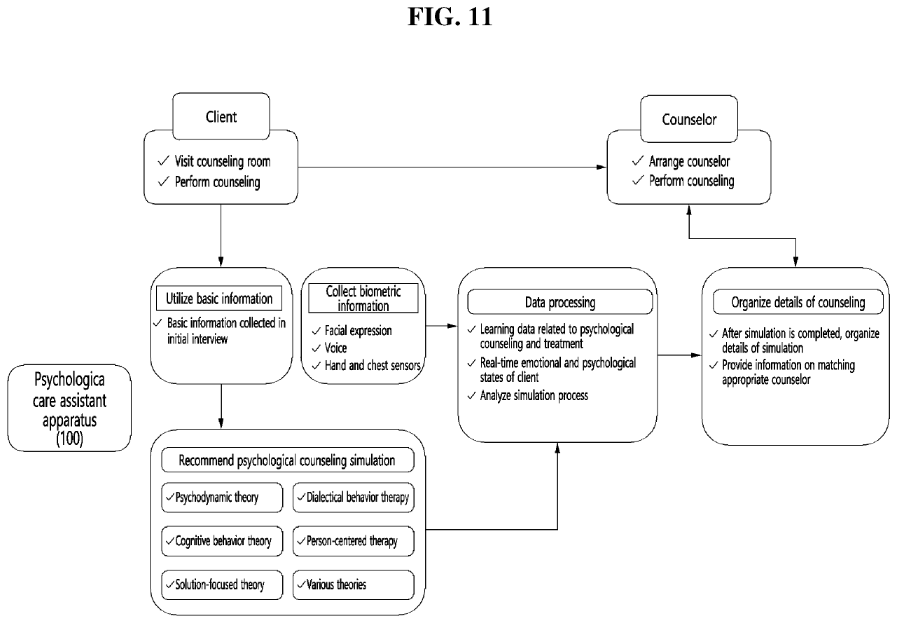

FIG. 11 is a view showing the concept of interactions between the apparatus 100 and a client and a counselor in a situation of assisting a therapeutic expert of individual and group psychological counseling (referred to as a “counselor” in the specification) according to an embodiment.

Referring to FIG. 11, the apparatus 100 may provide clients with a psychological education and psychological counseling simulation experience by utilizing the LMM-based psychological care assistant. At this point, question-and-answer information of the LMM-based psychological care assistant may be integrated with psychological diagnosis information generated on basis of the data collected and processed in the process of psychological test, initial intake interview, and psychological counseling. Based on this, the apparatus 100 may recommend a psychological education and psychological counseling simulation (e.g., psychodynamic theory, cognitive behavioral therapy, dialectical behavior therapy, person-centered therapy, solution-focused therapy, etc.) on the basis of psychological knowledge or theoretical orientation suitable for individual characteristics and major complaints of each client.

In addition, the apparatus 100 may provide the client with an experience of directly communicating with historically renowned psychologists such as Freud, Adler, Rogers, and the like. For example, the apparatus 100 may allow a digital human of the LMM-based psychological care assistant to interact with the client in the form of an avatar, and the avatar may be configured to have an appearance similar to that of a psychologist according to each theoretical orientation.

In addition, the apparatus 100 may select key topics during a simulation session of a predetermined period of time and provide the client with useful tasks based on the key topics after the simulation is completed. Data used in the psychological education and psychological counseling simulation for assisting the client is collected under the consent of participants, and all information can be processed safely.

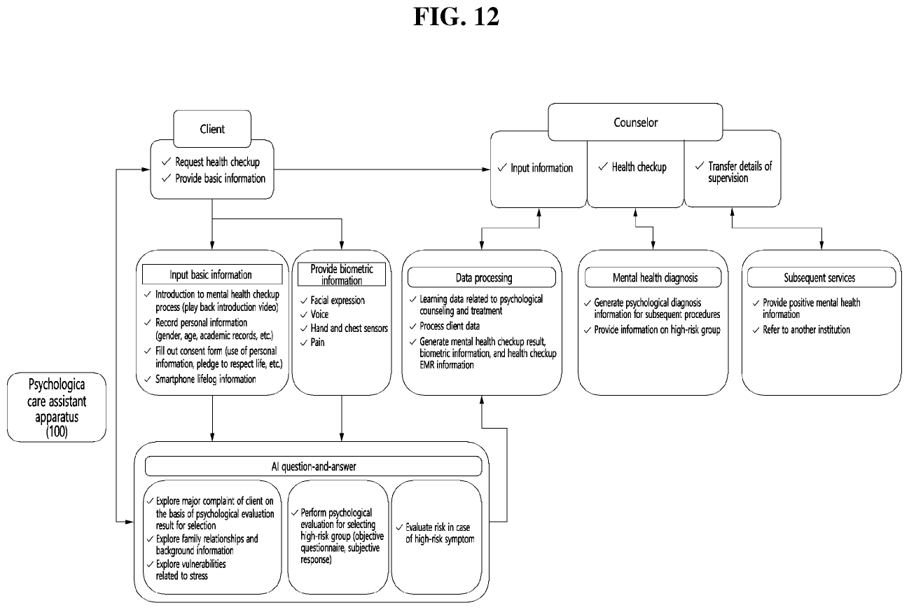

FIG. 12 is a view showing the concept of interactions between the apparatus 100 and a client and a counselor in a situation of assisting a health checkup specialist (referred to as a “counselor” in the specification) according to an embodiment.

Referring to FIG. 12, the apparatus 100 may utilize the LMM-based psychological care assistant to provide more effective counseling by detecting symptoms of psychological crisis through a psychological health checkup process and analyzing information related to resistance of the client.

The apparatus 100 may accurately evaluate the psychological state of a participant in the health checkup stage and provide an integrated psychological care service leading to counseling and treatment of an expert when necessary. At this point, provision of information may be performed by comprehensively utilizing biometric information and psychological tests, like in the case where the LMM-based psychological care assistant is utilized in an initial intake interview tool or psychological counseling and treatment.

For example, the apparatus 100 may provide psychological diagnosis information (R/O) and a positive mental health type by integrating a result of the mental health checkup of the client (e.g., 50 minutes per session), AI question-and-answers, and biometric information.

At this point, the apparatus 100 may provide current psychological diagnosis information of the client, together with data that is helpful for diagnosis (e.g., psychological evaluation, psychological care data, etc.), by integrating the data used for the AI question-and-answers and the expert.

For example, the apparatus 100 may provide data that can distinguish diseases having clear medical causes from embodied cognitions due to psychological problems, together with data on the high-risk group requiring medication (e.g., insomnia, hallucinations or delusions, symptoms of severe depression, etc.). In addition, an object of the present invention is to provide useful data in the process of diagnosing and treating patients to escalate positive mental health, and the apparatus 100 may help the patients to maintain better mental health, and provide various information needed for medical staff to accurately diagnose and plan an effective treatment.

According to the embodiment described above, as the correlation between verbal and nonverbal data is derived by comprehensively analyzing basic information and biometric information of a client, the present invention may enhance efficiency of the psychological health care by grasping psychological problems of the client in an early stage and implementing preventive interventions.

In addition, the present invention reduces the burden of work of psychotherapists, and expands accessibility to psychological care services by automatically generating a counseling and treatment plan customized for a client. Accordingly, as the dependency on the subjective determination of a counselor is lowered, and objective evaluation based on data is allowed, reliability and accuracy of psychological care service can be improved significantly.

Furthermore, the present invention may provide an environment in which a client may receive more continuous and personalized psychological support through automation and advancement in the techniques of psychological care, and practically contribute to promotion of psychological health and improvement of quality of life.

It should be understood that various embodiments of this document and the terms used herein are not intended to limit the technical features described in this document to specific embodiments, but include various modifications, equivalents, or substitutes of the embodiments. In connection with the description of drawings, similar reference numerals may be used for similar or related components. The singular form of a noun corresponding to an item may include one or more items, unless the related context clearly indicates otherwise.

In this document, each of phrases such as “A or B”, “at least one among A and B”, “at least either A or B”, “A, B, or C”, “at least one among A, B, and C”, and “at least either A, B, or C” may include all possible combinations of the items listed together in a corresponding phrase among the phrases. Terms such as “1st”, “2nd”, “first”, or “second” may be used only to distinguish a corresponding component from another corresponding component, and do not limit the components in any other aspect (e.g., importance or order). When a certain (e.g., a first) component is referred to as being “coupled” or “connected” to another (e.g., a second) component with or without a term such as “functionally” or “communicatively”, it means that the component may be connected to another component directly (e.g., wired), wirelessly, or through a third component.

The term “module” used in this document may include a unit implemented in hardware, software, or firmware, and may be used interchangeably with terms such as logic, logic block, part, or circuit. A module may be an integrally configured component, or a minimum unit of a component or a portion thereof that performs one or more functions. For example, according to an embodiment, a module may be implemented in the form of an application-specific integrated circuit (ASIC).

Various embodiments of this document may be implemented as software (e.g., a program) including one or more commands stored in a storage medium (e.g., a memory) that can be read by a device (e.g., an electronic device). The storage medium may include a random-access memory (RAM), a memory buffer, a hard drive, a database, an erasable programmable read-only memory (EPROM), an electrically erasable read-only memory (EEPROM), a read-only memory (ROM), and/or the like.

In addition, the processor in the embodiments of this document may call at least one command among one or more stored commands from the storage medium and execute the command. This allows the device to operate to perform at least one function according to the called at least one command. The one or more commands may include a code generated by a compiler or a code that can be executed by an interpreter. The processor may be a general-purpose processor, a Field Programmable Gate Array (FPGA), an Application Specific Integrated Circuit (ASIC), a Digital Signal Processor (DSP), and/or the like.

The storage medium that can be read by a device may be provided in the form of a non-transitory storage medium. Here, ‘non-transitory’ only means that the storage medium is a tangible device and does not include signals (e.g., electromagnetic waves), and this term does not distinguish the cases where data is stored semi-permanently on the storage medium from the cases where data is stored temporarily.

The method according to various embodiments disclosed in this document may be provided to be included in a computer program product. The computer program product may be traded between a seller and a buyer as goods. The computer program product may be distributed in the form of a machine-readable storage medium (e.g., a compact disc read only memory (CD-ROM)), or may be distributed online (e.g., downloaded or uploaded) through an application store (e.g., Play Store) or directly distributed between two user devices (e.g., smartphones). In the case of online distribution, at least a part of the computer program product may be at least temporarily stored in a machine-readable storage medium, such as a memory of a manufacturer's server, an application store's server, or a server, or may be temporarily generated.

According to various embodiments, each component (e.g., a module or a program) of the components described above may include a single or a plurality of entities. According to various embodiments, one or more of the components or operations of the components described above may be omitted, or one or more other components or operations may be added. Alternatively or additionally, a plurality of components (e.g., modules or a programs) may be integrated into a single component. In this case, the integrated component may perform one or more functions of each of the plurality of components in a way identical or similar to those performed by the corresponding component among the plurality of components before the integration. According to various embodiments, the operations performed by the modules, programs, or other components may be executed sequentially, in parallel, repeatedly, or heuristically, or one or more of the operations may be executed in a different order or omitted, or one or more other operations may be added.

### DESCRIPTION OF SYMBOLS

100: Apparatus
110: Memory
120: Processor
130: Input/Output Interface
140: Communication Interface

1. A method performed by a psychological care assistant apparatus operated by a processor, the method comprising:
an operation of acquiring basic information of a client;
an operation of acquiring biometric information of the client;
an operation of generating evaluation information about a psychological state of the client by utilizing an LMM model on the basis of the basic information and biometric information;
an operation of providing feedback information to the client or a counselor counseling the client on the basis of the evaluation information; and
an operation of acquiring an additional input on the counselor, and generating information on a psychological counseling or treatment plan by inputting a command corresponding to the additional input and the evaluation information into the LMM model.

2. The method according to claim 1, wherein the basic information includes personal information of the client, self-report psychological test results, and lifelog data.

3. The method according to claim 1, wherein the biometric information includes physiological information, behavioral information, vocal information, and verbal information.

4. The method according to claim 3, wherein the operation of acquiring biometric information includes an operation of storing a psychological factor of the client, which is determined by classifying a numerical or classification value of physiological information, behavioral information, vocal information, or verbal information corresponding to the biometric signal according to predefined criteria, to be mapped to the biometric information.

5. The method according to claim 4, wherein the operation of acquiring biometric information includes an operation of determining inconsistency between at least two pieces of information, among the physiological information, behavioral information, vocal information, and verbal information, corresponding to the biometric signal, determining that the acquired biometric signal is caused by a psychological resistance or false report of the client when the determined inconsistency exceeds a predetermined threshold, and reacquiring the biometric signal of the client.

6. The method according to claim 5, wherein the LMM model generates vector information combining the basic information and the biometric information on the basis of a self-attention mechanism or a cross-attention mechanism, and generates evaluation information about the psychological state of the client on the basis of the combined vector information.

7. The method according to claim 6, wherein the operation of generating evaluation information includes:
an operation of generating evaluation information about the psychological state of the client on the basis of the self-attention mechanism between text specifying the basic information and text defining the determined psychological factor; and
an operation of generating evaluation information about the psychological state of the client on the basis of the cross-attention mechanism between text specifying the basic information and an image or a video specifying the biometric information.

8. The method according to claim 1, wherein the feedback information includes temporal tendency in a change of the psychological state of the client and a warning about a psychologically high-risk group.

9. The method according to claim 1, wherein the additional input includes information on a current psychological state, details of previous counseling, and a result of observing a specific behavior or response, and the command corresponding to the additional input includes a command text for a specific task that the LMM model should perform on the basis of the additional input.

10. A psychological care assistant apparatus comprising:
a memory including instructions; and
a processor that performs a predetermined operation based on the instructions, wherein
the operation of the processor includes:
an operation of acquiring basic information of a client;
an operation of acquiring biometric information of the client;
an operation of generating evaluation information about a psychological state of the client by utilizing an LMM model on the basis of the basic information and biometric information;
an operation of providing feedback information to the client or a counselor counseling the client on the basis of the evaluation information; and
an operation of acquiring an additional input on the counselor, and generating information on a psychological counseling or treatment plan by inputting a command corresponding to the additional input and the evaluation information into the LMM model.
